(19) United States
(12) Patent Application Publication
(10) Pub. No.: US 2015/0045649 A1
(43) Pub. Date: Feb. 12, 2015

(54) BALLOON CATHETER AND A SYSTEM AND A METHOD FOR DETERMINING THE DISTANCE OF A SITE IN A HUMAN OR ANIMAL BODY FROM A DATUM LOCATION

(71) Applicant: FLIP TECHNOLOGIES LIMITED, Galway (IE)

(72) Inventors: John O'Dea, Galway (IE); Eoin Bambury, County Meath (IE); Adrian Mchugh, County Galway (IE)

(73) Assignee: FLIP TECHNOLOGIES LIMITED, Galway (IE)

(21) Appl. No.: 14/384,866

(22) PCT Filed: Mar. 15, 2013
(86) PCT No.: PCT/IE2013/000007

§ 371 Date: Sep. 12, 2014

(30) Foreign Application Priority Data
Mar. 15, 2012 (IE) S2012/0140

(51) Int. Cl. A61B 5/00; A61B 5/06; A61B 5/02; A61F 2/24; A61M 25/10

(52) U.S. Cl. CPC A61B 5/4851; A61F 2/2496; A61M 25/10; A61B 5/02; A61B 5/065; A61M 2025/0008
USPC 600/409; 600/508; 600/424

(57) ABSTRACT

In a system (2) having a balloon catheter (1) with a balloon (7) and an elongated catheter (3), a planimetry measuring system (19) in the balloon, and a linear distance measuring element (23) slideable along the elongated catheter from a proximal end (4) thereof to the datum location (8) when the balloon catheter has been inserted through the arterial system with the balloon located in the valve orifice (17), a plurality of secondary optically detectable elements (30) are equi-spaced longitudinally along the catheter and an optical encoder (32) in the linear distance measuring element counts the detectable elements as the linear distance measuring element is moved from a reset position to the datum location. A signal processor (20) reads signals from the linear distance measuring element and from the planimetry measuring system for determining the distance of the valve orifice of the aortic valve (9) from the datum location.

The present invention relates to a balloon catheter, and in particular, though not limited to a balloon catheter adapted for determining the distance of a site in a human or animal body from a datum location. The invention also relates to a system and a method for determining the distance of a site in a human or animal body from a datum location.

A percutaneous aortic valve replacement procedure, which is also known as a transcatheter aortic valve implantation (TAVI) procedure is a procedure performed by delivering a replacement valve using a catheter via the femoral artery. A number of challenges are associated with this procedure. For example, it is generally necessary to perform an aortic valvuloplasty as part of the valve replacement, whereby a dilation balloon is inflated inside the valve in order to release calcified valve cusps. It is also desirable to know the precise location of the valve, in order to allow accurate positioning of the replacement valve. It is also necessary to determine the size of the valve, in order that a replacement valve of the correct size is implanted. Additionally, it has been observed that localised calcification of the iliofemoral artery can result in complications arising during and after the procedure. The diameter of the iliofemoral artery may be measured using multi-detector computed tomographic angiography (MDCT) or other means. However, such MDCT diameter measuring apparatus tend to be relatively expensive and typically cost in excess of US$1,000,000, and furthermore, are not always available or utilised.

A recent study has shown that the risk of vascular complications is higher in patients with a minimal iliofemoral artery diameter which is smaller than the external diameter of the valve deployment catheter, in the presence of moderate or severe calcification, and in patients with peripheral vascular disease. The study concluded that a reduction from 8% to 1% in the rate of major complications has been observed during percutaneous aortic valve replacement procedures, if regions of local narrowing of the iliofemoral artery are known. However, it has been found that two types of narrowing of the iliofemoral artery may occur due to calcification. In one type the narrowed region of the artery remains compliant, while in the other type the narrowed area becomes quite rigid, and is not compliant. Where a narrowed region of an artery is compliant, the consequences resulting from narrowing of the artery are not as serious as when the narrowed region of the artery is not compliant, since once the narrowed region is compliant, the narrowed region expands to accommodate the valve deployment catheter as it passes through the narrowed region, while in the case of a non-compliant narrowed region, the narrowed region fails to expand to accommodate the valve deployment catheter. While currently known imaging modalities are capable of determining the diameter of narrowed regions in an artery, they are incapable of determining whether the narrowed region of the artery is compliant or non-compliant.

There is therefore a need for a device which addresses at least some of these issues, and provides a cost-effective solution to the problems being addressed.

The invention is directed towards providing a balloon catheter, and the invention is also directed towards a system and a method for determining the distance of a site in a human or animal body from a datum location.

According to the invention there is provided a balloon catheter comprising an elongated catheter, an inflatable balloon located on the catheter, and a linear distance measuring means located adjacent the catheter configured to measure linear distance on the catheter from the balloon.

In one aspect of the invention the linear distance measuring means is located on the catheter.

In another aspect of the invention the linear distance measuring means is configured to accommodate relative linear movement between the catheter and the linear distance measuring means.

Preferably, the linear distance measuring means is configured to accommodate linear movement of the catheter through the linear distance measuring means. Advantageously, the linear distance measuring means is slideably mounted on the catheter.

In another aspect of the invention the linear distance measuring means is responsive to relative linear movement between the catheter and the linear distance measuring means for determining the linear distance on the catheter from the balloon.

In another aspect of the invention a primary element is located on the catheter at a predefined distance from the balloon, the primary element defining a reset position of the linear distance measuring means.

In one embodiment of the invention the primary element is located at a predefined distance from one of the proximal end of the balloon and the distal end of the balloon. Preferably, the primary element is located adjacent the proximal end of the catheter. Alternatively, the primary element is located distally along the catheter between the proximal end of the catheter and the proximal end of the balloon.

In one aspect of the invention the primary element is located adjacent the proximal end of the balloon.

In another embodiment of the invention a detecting means is provided for detecting relative linear movement between the catheter and the linear distance measuring means, and for producing a signal indicative of the distance of the linear distance measuring means from the balloon. Preferably, the detecting means is located on the linear distance measuring means.

In another embodiment of the invention a plurality of longitudinally spaced apart secondary detectable elements are provided at predefined distances from the primary element and spaced apart therefrom, the secondary detectable elements being detectable by the detecting means for determining the distance of the linear distance measuring means from the balloon. Preferably, the secondary detectable elements are equi-spaced apart longitudinally on the catheter. Advantageously, the longitudinal spacing between the primary element and the adjacent one of the secondary detectable elements is similar to the spacing between the secondary detectable elements.

In another embodiment of the invention the primary element comprises a detectable element detectable by the detecting means.

In one aspect of the invention the detecting means is configured to count the ones of the secondary detectable elements detected by the detecting means as the one of the linear distance measuring means and the catheter is moved longitudinally relative to the other one of the linear distance measuring means and the catheter for producing a signal indicative of the distance of the linear distance measuring means along the catheter from the balloon. Preferably, the detecting means comprises an encoder. Advantageously, the encoder is configured to count the detected ones of the secondary detectable elements as the one of the linear distance measuring means and the catheter is moved longitudinally relative to the other one of the linear distance measuring means and the catheter.

In another aspect of the invention the secondary detectable elements comprise respective ones of optically detectable elements, magnetically detectable elements, and capacitively detectable elements, and the detecting means comprises one of an optical encoder, a magnetic encoder and a capacitive encoder.

In another embodiment of the invention at least each secondary detectable element comprises an encoded element with the distance of the secondary detectable element from the balloon being encoded therein.

In another embodiment of the invention each secondary detectable element comprises a barcode.

In another embodiment of the invention the primary element comprises one of an optically detectable element, a magnetically detectable element and a capacitively detectable element.

In another embodiment of the invention the primary element comprises an encoded element with the distance of the primary element from the balloon being encoded therein, the encoded element being readable by the detecting means.

In a further embodiment of the invention the encoded element of the primary element comprises a barcode.

In one embodiment of the invention each one of the primary and secondary detectable elements extends around the catheter in a band-like configuration. Preferably, each one of the primary and secondary detectable elements extends completely around the catheter.

In one embodiment of the invention at least the secondary detectable elements are printed onto the catheter by one of an optically detectable ink, a magnetic ink and an electrically conductive ink.

In another embodiment of the invention the primary element is printed onto the catheter by one of an optically detectable ink, a magnetic ink and an electrically conductive ink.

In a further embodiment of the invention a movement sensing means is located in the linear distance measuring means for detecting relative longitudinal movement between the linear distance measuring means and the catheter.

Preferably, the movement sensing means is configured to detect the direction of relative movement between the linear distance measuring means and the catheter, and to produce a signal indicative of the direction of the relative movement.

In one aspect of the invention the movement sensing means is configured to detect the distance moved by one of the linear distance measuring means and the catheter relative to the other one of the linear distance measuring means and the catheter.

Advantageously, the movement sensing means comprises a rotatable element rotatably mounted in the linear distance measuring means and configured to be in rotatable engagement with the catheter, and a rotary encoder co-operable with the rotatable element for monitoring the direction of rotation of the rotatable element and for producing a signal indicative of the direction of relative movement between the linear distance measuring means and the catheter.

Preferably, the rotary encoder of the movement sensing means is configured to determine distance of the linear distance measuring means from the balloon, and to produce a signal indicative of the linear distance along the catheter of the linear distance measuring means from the balloon.

In another embodiment of the invention the primary element comprises an abutment element engageable with the linear distance measuring means for defining the reset position of the linear distance measuring means.

In one embodiment of the invention a catheter accommodating bore extends through the linear distance measuring means, and the catheter is longitudinally slideable in the catheter accommodating bore.

In another embodiment of the invention the linear distance measuring means comprises a linear distance measuring element.

In a further embodiment of the invention the linear distance measuring element comprises a housing defining the catheter accommodating bore extending therethrough.

In a still further embodiment of the invention a balloon measuring means is provided for producing signals indicative of one of the diameter and the transverse cross-sectional area of the balloon adjacent a plurality of longitudinally spaced apart locations intermediate the respective opposite proximal and distal ends of the balloon. Preferably, the balloon measuring means comprises an impedance planimetry measuring means.

In another embodiment of the invention a pressure sensing means is provided for monitoring pressure within the balloon.

In one aspect of the invention the catheter extends from the proximal end to a distal end, and the balloon is located adjacent the distal end of the catheter.

In another aspect of the invention a bypass means is provided in the balloon catheter adjacent the balloon for accommodating a fluid in a lumen, a vessel or a valve in which the balloon is located from one of the proximal and distal ends of the balloon to the other one of the proximal and distal ends thereof. Preferably, the bypass means comprises a bypass conduit extending between and communicating a pair of ports, the ports being located with the balloon located therebetween. Advantageously, the bypass conduit extends through the catheter, and the respective ports are located on the catheter.

In one embodiment of the invention the bypass conduit extends through the balloon between a proximal end and a distal end thereof, the ports being located on the balloon adjacent the proximal end and the distal end thereof.

In another embodiment of the invention the bypass means comprises an elongated groove defined by the balloon extending from the proximal end to the distal end thereof.

The invention also provides a system comprising the balloon catheter according to the invention, and a signal processor configured to read signals from the linear distance measuring means of the balloon catheter and for determining the distance along the catheter of the linear distance measuring means from the balloon.

In one aspect of the invention the signal processor is configured to read signals from the balloon measuring means and for determining a location of the balloon at which one of the diameter and the cross-sectional area of the balloon is less than the corresponding ones of the diameters and the cross-sectional areas of the balloon adjacent the said location on respective opposite sides thereof with the balloon defining a waisted portion adjacent the said location.

In another aspect of the invention the signal processor is configured to determine the distance of the linear distance measuring means from the location of the balloon at which the balloon defines the waisted portion.

In a further aspect of the invention the signal processor is configured to produce a signal indicative of one or more of the distances along the catheter of the linear distance measuring means from the balloon, and the distance along the catheter of the linear distance measuring means from the location of the balloon at which the balloon defines the waisted portion.

Preferably, the signal processor is configured to produce a signal indicative of a representation of the longitudinal profile of the balloon.

Advantageously, the signal processor is configured to read signals from the pressure sensing means, and to determine the pressure within the balloon from the signals read from the pressure sensing means. Preferably, the signal processor is configured to produce a signal indicative of the value of the pressure in the balloon.

In one embodiment of the invention the signal processor is configured to compute a value of a distensibility index of a lumen, vessel or a valve orifice within which the balloon is located. Preferably, the signal processor is configured to compute the value of the distensibility index as a function of the transverse cross-sectional area of the balloon adjacent a location of the balloon adjacent which the value of the distensibility index of the lumen vessel or valve orifice is to be determined and the pressure within the balloon.

Advantageously, the signal processor is configured to produce a signal indicative of the value of the distensibility index.

In one embodiment of the invention the signals produced by the signal processor are adapted for applying to a visual display means for displaying data of which the signals are representative.

In one embodiment of the invention a visual display means is provided for displaying the data. Preferably, the visual display means is configured to display a representation of a longitudinal profile of the balloon.

In one embodiment of the invention the signal processor is configured to produce the signals adapted for applying to a laptop computer.

In another embodiment of the invention the signal processor comprises a microprocessor.

In another embodiment of the invention the signal processor comprises a computer.

Further the invention provides a method for determining the linear distance of a remote site in a vessel, lumen, valve or sphincter within a human or animal body from a datum location, the method comprising:

inserting the balloon catheter according to the invention into the human or animal body adjacent the datum location,

urging the balloon catheter through the human or animal body with the distal end thereof being the leading end until the balloon is located at the remote site,

inflating the balloon for retaining the balloon at the remote site,

with the linear distance measuring means adjacent the datum location, operating the signal processor of the system according to the invention to read signals from the linear distance measuring means and to determine the linear distance along the catheter of the linear distance measuring means from the balloon in order to determine the distance of the remote site from the datum location.

In one embodiment of the invention the balloon is inflated, and the signal processor is operated to read signals from the balloon measuring means and to determine a location of the balloon at which one of the diameter and the transverse cross-sectional area of the balloon is less than the corresponding ones of the diameters and the transverse cross-sectional areas of the balloon adjacent the said location on opposite sides thereof with the balloon defining a waisted portion adjacent the said location.

Preferably, the signal processor is operated to determine the linear distance along the catheter from the datum location to the location at which the balloon defines the waisted portion.

Advantageously, the balloon is inflated at the remote site until the balloon defines the waisted portion.

Preferably, the signal processor is operated to determine one of the diameter and the transverse cross-sectional area of the balloon adjacent the waisted portion defined by the balloon for determining one of the diameter and the cross-sectional area of the vessel, lumen, valve or sphincter adjacent the waisted portion of the balloon.

In one aspect of the invention the signal processor is operated to determine the pressure of inflating medium in the balloon.

In another aspect of the invention the signal processor is operated to compute a value of the distensibility index of the vessel, lumen, valve or sphincter adjacent the waisted portion of the balloon.

In another embodiment of the invention the remote site is a valve orifice in the human or animal body, and the balloon catheter is urged through the human or animal body until the balloon is located in the valve orifice and the balloon is inflated to define the valve orifice adjacent the waisted portion.

In one embodiment of the invention the valve orifice is a valve orifice of an aortic valve.

In another embodiment of the invention the remote site is a narrow region in a vessel or lumen, and the balloon catheter is urged through the human or animal body until the balloon is located in a narrow region of a vessel or lumen, and the balloon is inflated to define the narrow region adjacent the waisted portion.

The advantages of the invention are many. A particularly important advantage of the invention is that the balloon catheter and the system according to the invention allow the distance through an arterial system or other vascular system from a datum location, typically the point of entry into the arterial or vascular system to a remote site to be determined. By knowing the distance from the datum location to the remote site, a component can subsequently be accurately put in place at the remote site by, for example, a delivery catheter. This is a particularly important advantage when a valve of an aortic valve is being replaced in a transcatheter aortic valve implantation procedure.

A further advantage of the invention is that by knowing the distance from the datum location to the remote site, any other catheters, guide wires or the like which are to be subsequently inserted through the same arterial, vascular or other system to the remote site after removal of the balloon catheter can be accurately located at the remote site.

The advantage of providing the linear distance measuring element with both a means for detecting direction of relative linear movement between the linear distance measuring element and the catheter and for detecting the actual relative linear distance moved between the linear distance measuring element and the catheter, is that even if the linear distance measuring element is moved both distally and proximally as it is being moved from the reset position to the datum location, the distance between the remote site and the datum location will be relatively accurately determined by the signal processor.

A further advantage of the invention is that the balloon of the balloon catheter can be located in a desired location in the human or animal body without the need for use of radiation technology.

The invention will be more clearly understood from the following description of some preferred embodiments thereof, which are given by way of non-limiting examples, with reference to the accompanying drawings, in which:

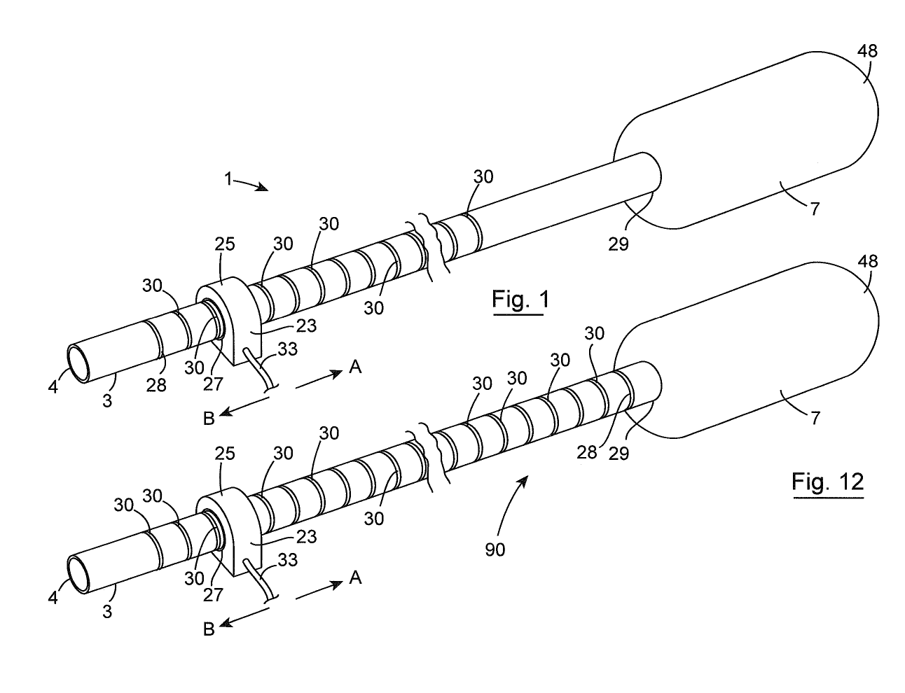

FIG. 1 is a perspective view of a balloon catheter according to the invention,

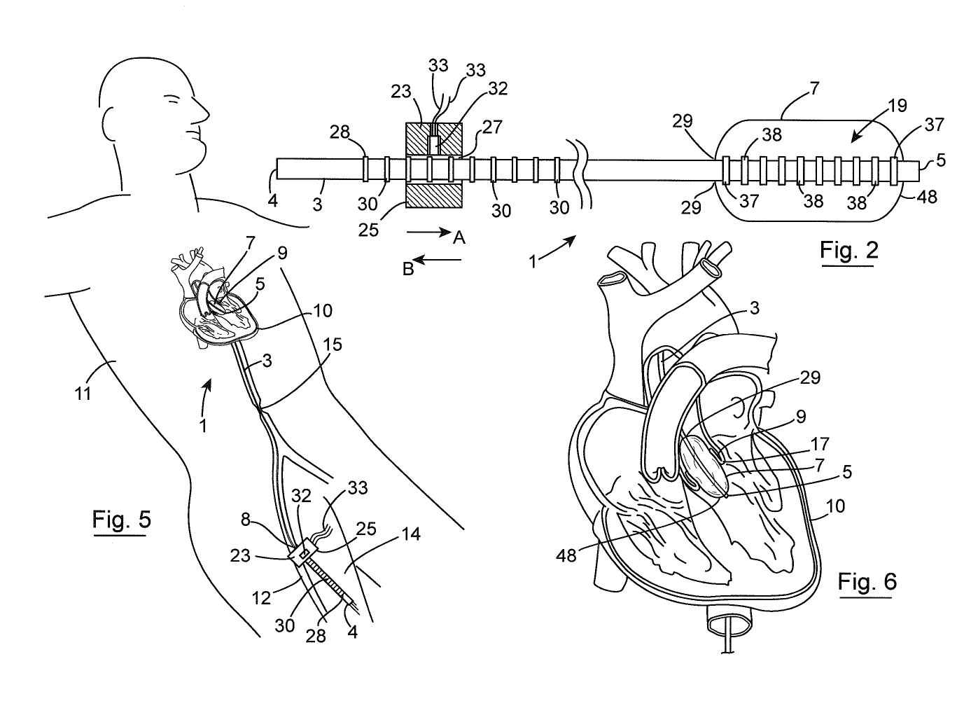

FIG. 2 is a partly cross-sectional view of the balloon catheter of FIG. 1,

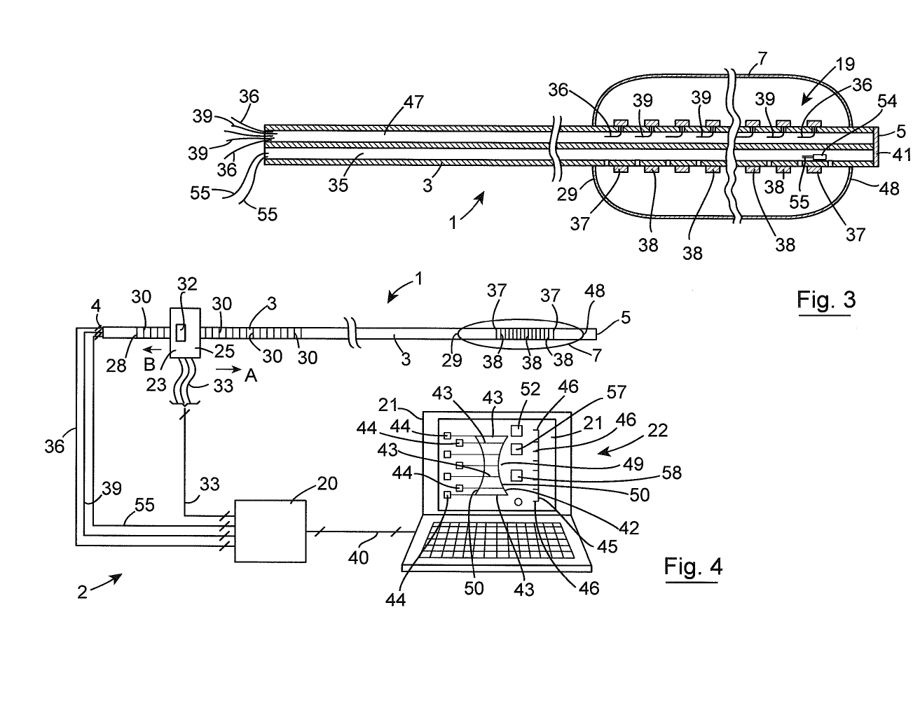

FIG. 3 is a cross-sectional view of the balloon catheter of FIG. 1,

FIG. 4 is a partly block representation of a system also according to the invention comprising the balloon catheter of FIG. 1,

FIG. 5 is a perspective view of the balloon catheter of FIG. 1 in use in a human subject,

FIG. 6 is an enlarged perspective view of a portion of the balloon catheter of FIG. 1 also in use in the human subject of FIG. 5,

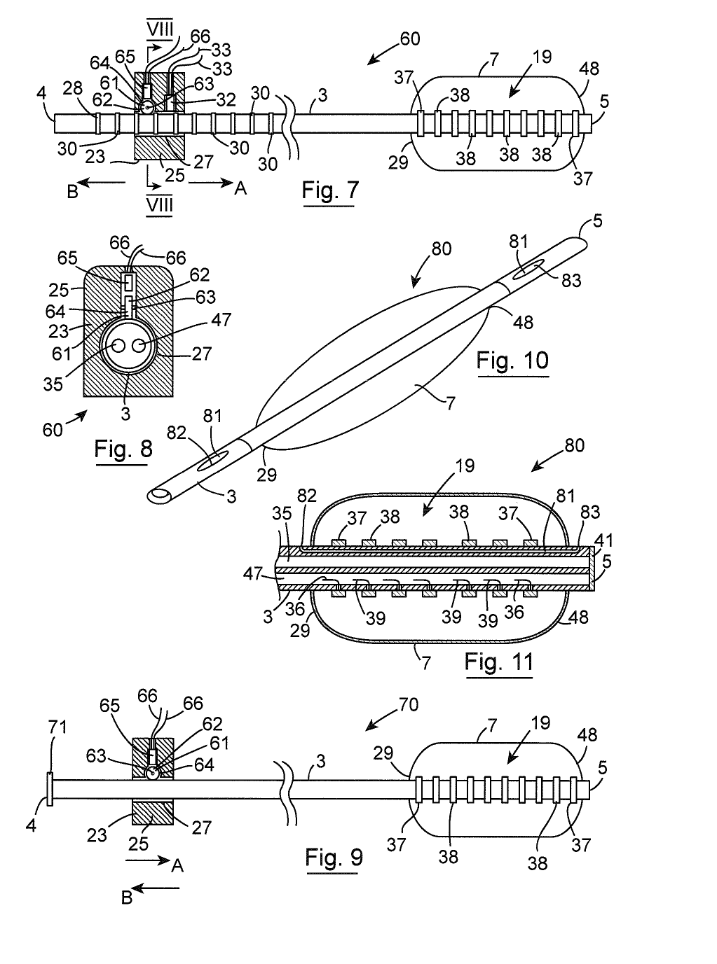

FIG. 7 is a partly cross-sectional side elevational view of a balloon catheter according to another embodiment of the invention,

FIG. 8 is a cross-sectional end elevational view on the line VIII-VIII of FIG. 7 of the balloon catheter of FIG. 7,

FIG. 9 is a partly cross-sectional side elevational view of a balloon catheter according to another embodiment of the invention,

FIG. 10 is a perspective view of a portion of a balloon catheter according to a further embodiment of the invention,

FIG. 11 is a cross-sectional side elevational view of the portion of the balloon catheter of FIG. 11, and

FIG. 12 is a side elevational view of a balloon catheter according to another embodiment of the invention.

Referring to the drawings and initially to FIGS. 1 to 6, there is illustrated a balloon catheter according to the invention, indicated generally by the reference numeral 1, for use in a system also according to the invention and indicated generally by the reference numeral 2 for determining linear distance from a datum location to a site within a human or animal body. The balloon catheter 1 and the system 2 are also adapted for determining one of the transverse cross-sectional area of a lumen, vessel, valve orifice or sphincter, and for determining a value of a distensibility index of a lumen, vessel, valve orifice or sphincter, as will be described below. The balloon catheter 1 comprises an elongated catheter 3 extending between a proximal end 4 and a distal end 5, and an inflatable balloon 7 located on the catheter 3 adjacent the distal end 5 thereof.

In this embodiment of the invention the balloon catheter 1 and the system 2 are adapted for use in a transcatheter aortic valve implantation procedure, and the balloon catheter 1 and the system 2 are adapted for determining the linear distance from a datum location 8 to an aortic valve 9, the subject of the transcatheter aortic valve implantation procedure in the heart 10 of a human body 11 along the catheter 3, see FIGS. 5 and 6. In this embodiment of the invention the balloon catheter 1 is adapted to be entered into the arterial system of the human body 11 through the iliofemoral artery 12 adjacent one of the legs 14 of the human body 11, and the datum location 8 is the point of entry in the leg 14 through which the balloon catheter 1 is entered through the leg 14 into the iliofemoral artery 12. The balloon catheter 1 and the system 2 are also adapted to determine the distance from the datum location 8 to any restricted or narrow regions in the iliofemoral artery 12, for example, the restricted and narrow region 15.

The balloon catheter 1 and the system 2 are also adapted to determine either or both of the transverse cross-sectional area and the diameter of the valve orifice 17 of the aortic valve 9, and of the restricted or narrow region 15 of the iliofemoral artery 12 between the datum location 8 and the aortic valve 9. Further, the balloon catheter 1 and the system 2 are adapted to determine the distensibility of the valve orifice 17 of the aortic valve 9, and of the restricted or narrow regions 15 of the iliofemoral artery 12, by determining respective values of the distensibility indices of the valve orifice 17 and the narrow region 15.

The balloon catheter 1 is adapted for entering into the cardiovascular system through the iliofemoral artery 12 in the leg 14 of a subject, and the balloon 7 is adapted for locating in the valve orifice 17 of the aortic valve 9 for dislodging the diseased valve. The balloon catheter 1 with the balloon 7 located in the valve orifice 17 of the aortic valve 9 and inflated therein is adapted for determining the linear distance of the aortic valve 9 along the catheter 3 from the datum location 8. A balloon measuring means, namely, an impedance planimetry measuring system 19, which is described below, is located in the balloon 7 for producing signals indicative of one or both of the transverse cross-sectional area and the diameter of the balloon 7 at predefined longitudinally spaced apart locations along the balloon 7, for in turn determining the area of the valve orifice 17 of the aortic valve 9 for in turn determining the size of a suitable replacement valve.

The system 2 according to the invention comprises a signal processing means, namely, a signal processor 20 which is configured to read signals from the impedance planimetry measuring system 19 in the balloon 7 and to determine either or both the transverse cross-sectional area and the diameter of the balloon 7 at the predefined longitudinally spaced apart locations from the signals, and to produce signals for applying to a visual display means, namely, a visual display screen 21 of a laptop computer 22 for displaying a graphical representation of a longitudinal profile of the inflated balloon 7 on the screen 21, and also for displaying one or both of the transverse cross-sectional areas and the diameters of the balloon 7, which in this case is the diameters of the balloon 7 at the respective predefined longitudinally spaced apart locations along the balloon 7. In this embodiment of the invention the signal processor 20 is also configured to compute a value of the distensibility index of the valve orifice 17 of the aortic valve 9 and to produce a signal indicative of the value of the distensibility index for applying to the laptop computer 22 for displaying the distensibility index value on the visual display screen 21.

A linear distance measuring means comprising a linear distance measuring element 23 is slideably located on the catheter 3 for measuring linear distance along the catheter 3. The linear distance measuring element 23 comprises a measuring housing 25 having a catheter accommodating bore 27 extending therethrough for slideably accommodating the catheter 3 through the measuring housing 25. A primary element, which in this case is provided as a primary optically detectable element comprises a primary optically detectable band 28 located on and extending around the catheter 3 towards the proximal end 4 thereof. The primary optically detectable band 28 is located on the catheter 3 at a predefined known linear distance along the catheter 3 from a proximal end 29 of the balloon 7. A plurality of longitudinally equi-spaced apart secondary detectable elements provided by respective secondary optically detectable bands 30 are located on and extend around the catheter 3 distally from the primary optically detectable band 28. The spacing between the primary optically detectable band 28 and the adjacent one of the secondary optically detectable bands 30 is similar to the spacing between the secondary optically detectable bands 30. Accordingly, the secondary optically detectable bands 30 are located at respective predefined known distances from the primary optically detectable band 28, and in turn from the proximal end 29 of the balloon 7.

A detecting means, which in this embodiment of the invention comprises an optical encoder 32, is mounted in the measuring housing 25 of the linear distance measuring element 23 for detecting the primary optically detectable band 28 and the secondary optically detectable bands 30. The optical encoder 32 is configured to produce a signal indicative of the count of the number of secondary optically detectable bands 30 detected by the optical encoder 32 as the linear distance measuring element 23 is urged distally along the catheter 3 in the direction of the arrow A from a reset position adjacent the proximal end 4 of the catheter 3 on the proximal side of the primary optically detectable band 28, past the primary optically detectable band 28. Since the secondary optically detectable bands 30 are equi-spaced apart from each other and of known distances from the primary optically detectable band 28, the signal indicative of the count of the secondary optically detectable bands 30 produced by the optical encoder 32 as the linear distance measuring element 23 is urged distally in the direction of the arrow A along the catheter 3 from the reset position past the primary optically detectable band 28 is indicative of the distance of the location of the linear distance measuring element 23 from the primary optically detectable band 28. Since the primary optically detectable band 28 is located at a known distance from the proximal end 29 of the balloon 7, the signal produced by the optical encoder 32 which is indicative of the count of the secondary optically detectable bands 30 is also indicative of the location of the linear distance measuring element 23 from the proximal end 29 of the balloon 7. The linear distance of the location of the measuring element 23 from the proximal end 29 of the balloon 7 is obtained by subtracting the distance between the measuring element 23 and the primary optically detectable band 28 from the length of the catheter 3 between the primary optically detectable band 28 and the proximal end 29 of the balloon 7.

The primary and secondary optically detectable bands 28 and 30 are formed on the catheter 3 by a suitable printing process. The distance along which the secondary optically detectable bands 30 are provided along the catheter 3 depends on the function for which the balloon catheter 1 is provided. In some cases the secondary optically detectable bands 30 may be located along the entire length of the catheter 3 from the primary optically detectable band 28 to the proximal end 29 of the balloon 7. However, in general, the secondary optically detectable bands 30 will be located on the catheter 3 over a distance from the primary optically detectable band 28 which would be greater than the length of the catheter 3 which would normally extend outwardly from the datum location through which the balloon catheter 1 is entered into the human or animal body. The precision with which the linear distance along the catheter 3 of the linear distance measuring element 23 from the balloon 7 can be measured depends on the spacing between the secondary optically detectable bands 30. The closer the secondary optically detectable bands 30 are to each other, the higher the precision with which the distance of the linear distance measuring element 23 from the proximal end 29 of the balloon 7 can be measured. In cases where the secondary optically detectable bands 30 are provided over the entire length of the catheter 3 from the proximal end 4 thereof to the proximal end 29 of the balloon 7, it is envisaged that the primary optically detectable band 28 would be the first of the bands 28 and 30 located towards the proximal end 29 of the balloon 7, and as will be described below with reference to the balloon catheter of FIG. 12, the measuring element 23 would be located adjacent the datum location 8 at the point of entry of the balloon catheter into the arterial system of the human body, and as the catheter is being urged into the arterial system through the measuring element 23, the count of the secondary optically detectable bands 30 would be directly indicative of the distance of the measuring element 23 from the proximal end 29 of the balloon 7.

A first communicating means comprising electrically conductive wires 33 extending from the optical encoder 32 through the measuring housing 25 accommodate signals of the count of the secondary optically detectable bands 30 to the signal processor 20. The signal processor 20 is configured to read the signals from the optical encoder 32 and to compute the linear distance along the catheter 3 of the linear distance measuring element 23 from the proximal end 29 of the balloon 7 from the signals read from the optical encoder 32. The computed linear distance is stored in the signal processor 20.

Returning now to the balloon catheter 1, an inflating lumen 35 extends through the catheter 3 from the proximal end 4 thereof and communicates with the balloon 7 for accommodating an inflating medium for inflating the balloon 7. In this embodiment of the invention the inflating medium is an electrically conductive medium, and typically, is a saline solution.

The impedance planimetry measuring system 19 comprises a pair of stimulating band electrodes 37 located on the catheter 3 within the balloon 7 and longitudinally spaced apart from each other. A plurality of longitudinally equi-spaced apart sensing band electrodes 38 are also located on the catheter 3 within the balloon 7 and between and spaced apart from the stimulating electrodes 37. Second communicating means comprising electrically conductive wires 36 and 39 are accommodated through a wire accommodating lumen 47 extending through the catheter 3 from the balloon 7 to the proximal end 4 of the catheter 3, and extend through the proximal end 4 of the catheter 3 to the signal processor 20, for accommodating electrical signals between the stimulating and sensing electrodes 37 and 38, respectively, on the one hand, and the signal processor 20 on the other hand. An end cap 41 sealably closes the inflating lumen 35 and the wire accommodating lumen 47 at the distal end 5 of the catheter 3.

The signal processor 20 is configured to output a constant current signal to the stimulating electrodes 37, and to read resulting voltage signals from the sensing electrodes 38 when the balloon 7 is inflated with the electrically conductive medium for in turn determining the diameter of the balloon 7 at locations adjacent the respective sensing electrodes 38. The voltage signals read from the sensing electrodes 38 are indicative of both the transverse cross-sectional area and the diameter of the balloon 7 adjacent the respective corresponding sensing electrodes 38. The signal processor 20 is configured to compute both the transverse cross-sectional area and the diameter of the balloon 7 adjacent the sensing electrodes 38 from the signals read from the sensing electrodes 38. The computation of the transverse cross-sectional area and the diameter of the balloon 7 adjacent the respective sensing electrodes 38 from voltage signals read from sensing electrodes of such an impedance planimetry measuring system is described in PCT published Application Specification No. WO 2009/001328 of the present applicant, and further description should not be required. In the computation of the diameter of the balloon 7 adjacent the respective sensing electrodes 38, it is assumed that the balloon 7 when inflated is of circular transverse cross-sectional area, and the diameter of the balloon 7 is derived from the computed transverse cross-sectional area.

The signal processor 20 is configured to produce data signals indicative of a graphical representation of a longitudinal profile of the inflated balloon 7 from the computed values of the respective transverse cross-sectional areas and the diameters of the inflated balloon 7 adjacent the respective sensing electrodes 38. The signal processor 20 outputs data signals indicative of the graphical representation of the longitudinal profile of the inflated balloon 7 to the laptop computer 22 through a data bus 40 which in turn displays an image 42 of the graphical representation of the longitudinal profile of the inflated balloon 7 on the visual display screen 21, see FIG. 4. Lines 43 representative of the axial locations of the sensing electrodes 38 relative to the balloon 7 are also displayed on the visual display screen 21 superimposed on the image 42 of the inflated balloon 7. Windows 44 corresponding to the lines 43 are also displayed on the visual display screen 21. The computed numerical values of the diameter of the balloon 7 adjacent the respective sensing electrodes 38 are displayed in the corresponding windows 44.

A scale 45 with graduations 46 numbered from zero upwardly represents the length of the balloon 7 in millimetres, so that any location longitudinally along the image 42 representative of the inflated balloon 7 can be readily identified. The graduation zero represents the proximal end 29 of the balloon 7.

On the balloon 7 being inflated in the valve orifice 17 of the aortic valve 9, the action of the valve orifice 17 on the inflated balloon 7 results in the inflated balloon 7 being waisted intermediate the proximal end 29 and a distal end 48 of the balloon 7. The waist of the inflated balloon 7 is represented by a waisted portion 49 of the image 42 of the inflated balloon 7 on the visual display screen 21, see FIG. 4. Thus, the waisted portion 49 of the image 42 is representative of the precise location of the valve orifice 17 of the aortic valve 9 relative to the balloon 7.

The signal processor 20 is configured to identify the waisted portion 49 of the image 42 by, for example, determining the slopes of the respective opposite lines 50 defining the longitudinal profile of the inflated balloon 7, by curve fitting of those lines 50, or by determining the minimum diameter of the image 42 of the balloon 7, see FIG. 4. Such techniques will be well known to those skilled in the art. The signal processor 20 is configured so that on identifying the waisted portion 49 of the image 42, the signal processor 20 then determines the linear distance of the waisted portion 49 along the catheter 2 from the proximal end 29 of the balloon 7. The signal processor 20 is configured to sum this computed distance between the waisted portion 49 and the proximal end 29 of the balloon 7 to the already computed and stored distance between the proximal end 29 of the balloon 7 and the linear distance measuring element 23 when the linear distance measuring element 23 is abutting the leg 14 of the subject adjacent the datum location 8 in order to produce the linear distance of the valve orifice 17 of the aortic valve 9 from the datum location 8. This computed distance is then displayed in a window 52 of the visual display screen 21.

A pressure sensing means, in this embodiment of the invention a pressure sensor 54 measures the pressure of the inflating medium within the balloon 7. The pressure sensor 54 is located in the inflating lumen 35 of the catheter 3 within the balloon 7, and effectively directly detects the pressure of the inflating medium in the balloon 7. A third communicating means, namely, wires 55 from the pressure sensor 54 extends through the inflating lumen 35 of the catheter 3, and extend through the proximal end 4 of the catheter 3 from the inflating lumen 35 to the signal processor 20. The signal processor 20 is configured to read signals from the pressure sensor 54 and to determine the pressure of the inflating medium within the balloon 7 from the read signals, and to produce a signal indicative of the value of the pressure of the inflating medium within the balloon 7. The signal indicative of the value of the pressure of the inflating medium within the balloon 7 is applied to the laptop computer 22 through the data bus 40 for display in a window 57 of the visual display screen 21.

The signal processor 20 is configured to compute from the value of the pressure of the inflating medium within the balloon 7, the value of the distensibility index for the valve orifice 17 of the aortic valve 9 within which the balloon 7 is located or a restricted or narrow region 15 of an artery 12 within which the balloon 7 is located. The distensibility index value of the aortic valve is computed by the signal processor 20 by dividing the computed transverse cross-sectional area of the waisted portion 49 of the balloon 7, which corresponds to the transverse cross-sectional area of the valve orifice 17 of the aortic valve 9 by the computed value of the pressure of the inflating medium within the balloon 7. The distensibility index of the narrow region 15 is similarly determined by dividing the computed cross-sectional area of the neck of the narrow region by the computed value of the pressure of the inflating medium in the balloon 7. The computation of distensibility indices is described in U.S. published Patent Application No. 2010/0305479-A1.

The signal processor 20 is configured to produce a signal indicative of the value of the distensibility index which is outputted to the laptop computer 22, and displayed in a window 58 in the visual display screen 21. The linear distance along the catheter 3 of the valve orifice 17 of the aortic valve 9 and each restricted or narrow region 15 of the artery 12, such as the iliofemoral artery 12 from the datum point 8 which is computed by the signal processor 20 is displayed in the window 52 on the display screen 21.

The signal processor 20 may be any suitable signal processor, for example, a microprocessor, a logic controller, or any other signal processor.

In use, with the balloon 7 of the balloon catheter 1 deflated, the balloon catheter 1 with the distal end 5 leading is entered through the datum location 8 in the leg 14 of the subject into the iliofemoral artery 12, and is urged through the iliofemoral artery 12 into the cardiovascular system until the balloon 7 is located in the valve orifice 17 of the aortic valve 9. Prior to entering the balloon catheter 1 into the iliofemoral artery 12 and in turn through the cardiovascular system to the aortic valve 9, a guide wire (not shown) is inserted through the datum location 8 in the leg 14 of the subject through the iliofemoral artery 12 and the cardiovascular system to the valve orifice 17 of the aortic valve 9. The balloon catheter 1 is then urged over the guide wire until the balloon 7 is located in the valve orifice 17 of the aortic valve 9. This aspect of the location of the balloon of a balloon catheter in a valve orifice or any other remote location in a human or animal body will be well known to those skilled in the art. The balloon 7 is then inflated with the electrically conductive saline solution through the inflating lumen 35.

Additionally, when the balloon 7 of the balloon catheter 1 is initially engaged in the valve orifice 17 of the aortic valve 9, the distal end 5 of the balloon catheter 1 displaces the valve leaves from the valve orifice 17. The balloon 7 is then located in the valve orifice 17 and inflated with the electrically conductive medium in order to dilate the valve orifice 17 of the aortic valve 9 and to dislodge and release any calcified valve cusps in the valve orifice 17. On dislodging of calcified cusps from the valve orifice 17, the balloon 7 is again inflated with the electrically conductive medium until the valve orifice 17 is just about to dilate.

A stimulating current signal is applied to the stimulating electrodes 37 under the control of the signal processor 20. Voltage signals from the sensing electrodes 38 are read by the signal processor 20, which in turn determines the values of the transverse cross-sectional area and the diameter of the inflated balloon adjacent the respective sensing electrodes 38. The data signals indicative of the graphical representation of the longitudinal profile of the inflated balloon 7 are computed by the signal processor 20 and are outputted to the laptop computer 22, which in turn displays the image 42 of the longitudinal profile of the inflated balloon 7 on the visual display screen 21, with the lines 43 which are representative of the sensing electrodes 38 superimposed on the image 42. The computed values of the diameter of the inflated balloon 7 are displayed in the windows 44 on the display screen 21 adjacent the corresponding lines 43 which are representative of the sensing electrodes 38. The diameter of the valve orifice 17 can be read from the one of the windows 44 which corresponds to the line 43 adjacent the waisted portion 49 of the image 42 of the inflated balloon 7 on the visual display screen 21. From this value, the appropriate size of a replacement valve can be determined.

The signal processor 20 identifies the waisted portion 49 of the balloon 7 and computes its linear distance from the proximal end 29 of the inflated balloon 7. The computed linear distance between the waisted portion 49 of the balloon 7 and the proximal end 29 of the balloon 7 is stored.

The linear distance measuring element 23 is urged along the catheter 3 in the direction of the arrow B to the reset position at the proximal end 4 of the catheter 3 to the proximal side of the primary optically detectable band 28. From the reset position, the linear distance measuring element 23 is urged along the catheter 3 distally in the direction of the arrow A towards the datum location 8 until the linear distance measuring element 23 abuts the leg 14 of the subject adjacent the datum location 8. While the linear distance measuring element 23 is being urged along the catheter 3 in the direction of the arrow A from the reset position to the datum location 8, the signal processor 20 reads signals from the optical encoder 32 in the linear distance measuring element 23, which are indicative of the count of the secondary optically detectable bands 30, and from the signals read from the optical encoder 32, the signal processor 20 computes the distance between the measuring element 23 and the primary optically detectable band 28, which is the distance between the datum location 8 and the primary optically detectable band 28. This computed distance is stored in the signal processor 20. The signal processor 20 then computes the distance of the datum location 8 from the proximal end 29 of the balloon 7 by subtracting the stored computed distance between the datum location 8 and the primary optically detectable band 28 from the length of the catheter 3 between the primary optically detectable band 28 and the proximal end 29 of the balloon 7. This computed distance between the datum location 8 and the proximal end 29 of the balloon 7 is then stored and added to the stored value of the distance of the waisted portion 49 of the image 42 of the inflated balloon 7 from the proximal end 29 of the inflated balloon 7, in order to produce the value of the distance of the valve orifice 17 of the aortic valve 9 from the datum location 8. The computed value of the distance of the valve orifice 17 of the aortic valve 9 from the datum location 8 is then displayed in the window 52 of the visually display screen 21. The order in which the distance of the waisted portion 49 of the inflated balloon 7 from the proximal end 29 of the balloon 7 and the distance of the datum location 8 from the proximal end 29 of the balloon 7 are computed may be reversed.

Accordingly, when the balloon catheter 1 is withdrawn, and replaced by a valve deployment catheter which delivers the replacement valve for placing in the valve orifice 17 of the aortic valve 9, a surgeon knows the precise distance the valve deployment catheter must be entered from the datum location 8 in the leg 14 of the subject through the iliofemoral artery 12 and the cardiovascular system in order to accurately locate the replacement valve in the valve orifice 17 of the aortic valve 9.

The signal processor 20 determines the pressure of the inflating medium within the balloon 7 from the signals read from the pressure sensor 54, which is then displayed in the window 57 on the visual display screen 21.

The value of the distensibility index of the valve orifice 17 of the aortic valve 9 is determined by the signal processor 20 as follows. The balloon 7 of the balloon catheter 1 is inflated in the valve orifice 17, until the valve orifice 17 just commences to dilate. With the valve orifice 17 in the just dilated state, the transverse cross-sectional area of the valve orifice 17 is determined by the signal processor 20 from signals read from the sensing electrode 38 or the sensing electrodes 38 adjacent the valve orifice 17. The corresponding pressure of the inflating medium in the balloon 7 as the valve orifice 17 just commences to dilate is read from the pressure sensor 54 by the signal processor 20. The signal processor 20 computes the distensibility index value of the valve orifice 17 by dividing the computed transverse cross-sectional area of the valve orifice 17 in the just dilated state by the corresponding pressure of the inflating medium within the balloon 7. The computed value of the distensibility index is displayed in the window 58 of the visual display screen 21. The distensibility index of the valve orifice 17 may be determined by other relationships between area and/or diameter of the valve orifice on the one hand, and the pressure of the inflating medium in the balloon 7 on the other hand, as are described in U.S. published Patent Application No. 2010/0305479-A1.

Additionally, as the balloon catheter 1 is being withdrawn through the cardiovascular system and the iliofemoral artery 12, the balloon 7 is inflated sufficiently to allow monitoring of the transverse cross-sectional area of the iliofemoral artery 12 and other cardiovascular arteries in order to allow identification of regions 15 of the arteries which are restricted or narrowed due to calcification of the artery or due to other reasons. The distances of these restricted or narrowed regions 15 of the arteries from the datum location 8 in the leg 14 of the subject are computed in similar manner as described with reference to the computation of the distance of the valve orifice 17 of the aortic valve 9 from the datum location 8. The diameters of the restricted or narrowed regions 15 of the arteries are computed as the balloon catheter 1 is being withdrawn, and the diameters are displayed in the windows 44 corresponding to the sensing electrodes 38. The waisted portion 49 of the balloon represents the minimum diameter of each restricted or narrow region 15 of the relevant artery. The distance of the waisted portion of each narrow region 15 from the datum location 8 as the balloon catheter 1 is being withdrawn through the cardiovascular and the arterial system is displayed in the window 52 of the visual display screen 21.

Additionally, the distensibility index value of each restricted or narrowed region 15 of the arteries is determined by further inflating the balloon 7 in the restricted or narrow region 15 of the relevant artery until the restricted or narrow region 15 commences to dilate. The distensibility index value is then computed by dividing the transverse cross-sectional area of the waisted portion 49 of the balloon 7 as the restricted or narrow region of the artery commences to dilate by the corresponding pressure of the inflating medium within the balloon 7 read from the pressure sensor 54. The computed distensibility index value is displayed in the window 58 of the visual display screen. The distensibility index of the narrow region 15 may be determined by other relationships between area, and/or diameter of the narrow region on the one hand and the pressure of the inflating medium in the balloon 7 on the other hand, as described in U.S. published Patent Application No. 2010/0305479-A1.

Additionally, the balloon catheter 1 may be used to dilate some or all of the restricted or narrow regions 15 of the arteries by inflating the balloon 7 to dilate the restricted or narrow regions 15. In particular, it is envisaged that the restricted or narrow regions 15 which have a distensibility index value indicative of a relatively compliant restricted or narrow region 15 would be dilated by the balloon 7 of the balloon catheter 1, although less compliant restricted or narrow regions 15 could also be dilated by the balloon 7 of the balloon catheter 1.

By determining the distensibility index value of the relevant artery adjacent each restricted or narrow region 15 thereof, since the distensibility index value gives an indication of the compliability of the artery adjacent the restricted or narrow region 15, a surgeon can then make a decision as to which of the restricted or narrow regions 15 are suitable for dilation by the balloon 7 of the balloon catheter 1.

It is envisaged that an audible or visual alarm may be provided to indicate when the inflated balloon encounters a calcification restricted or narrowed region in the iliofemoral artery or in any of the cardiovascular or other arteries. Indeed, it is envisaged that the balloon 7 may be inflated or partially inflated as the balloon catheter 1 is being urged through the illiofemoral artery to the aortic valve for detecting such calcification restricted narrow regions, and the cardiologist would be alerted to the presence of such restricted or narrowed regions by the alarm. The alarm could also be used to indicate when the balloon was in the valve orifice of the aortic valve, if the balloon was inflated or partly inflated as the balloon was approaching the valve orifice.

Referring now to FIGS. 7 and 8, there is illustrated a balloon catheter according to another embodiment of the invention, indicated generally by the reference numeral 60. The balloon catheter 60 is substantially similar to the balloon catheter 1, and similar components are identified by the same reference numerals. The balloon catheter 60 is also adapted for use in the system 2. The main difference between the balloon catheter 60 and the balloon catheter 1 is in the linear distance measuring element 23. As well as comprising a detecting means, which in this embodiment of the invention is also provided by an optical encoder 32 for detecting the primary and secondary optically detectable bands 28 and 30, the linear distance measuring element 23 also comprises a movement sensing means which comprises a rotatably mounted element 61 provided by a circular disc 62 which is rigidly mounted on a shaft 63 rotatably mounted in the measuring housing 25. The disc 62 defines a peripheral circumferential surface 64 which extends into the catheter accommodating bore 27 to rotatably engage the catheter 3 of the balloon catheter 1 and rotate in response to and in a rotational direction corresponding to the direction of relative linear movement between the linear distance measuring element 23 and the catheter 3.

A rotary encoder 65 mounted in the linear distance measuring element 23 monitors rotation and in particular the direction of rotation of the rotatable element 61 and produces a signal indicative of the direction of relative movement between the linear distance measuring element 23 and the catheter 3. Wires 66 from the rotary encoder 65 communicate signals therefrom to the signal processor 20 of the system 2.

The signal processor 20 is configured to read the signals from the optical encoder 32 as already described with reference to the system 2 of FIGS. 1 to 6, and from the rotary encoder 65 in order to determine the distance travelled by the linear measuring element 23 along the catheter 3. The signal processor 20 is programmed to sum the count of the secondary optically detectable bands 30 detected by the optical encoder 32 while the signals from the rotary encoder 65 are indicative of the direction of movement of the linear distance measuring element 23 along the catheter 3 being in a distal direction, namely, in the direction of the arrow A, and to subtract the count of the secondary optically detectable bands 30 detected by the optical encoder 32 in response to signals from the rotary encoder 65 being indicative of movement of the linear distance measuring element 23 along the catheter 3 in a generally proximal direction, namely, in the direction of the arrow B.

The advantage of providing the movement sensing means in the linear distance measuring element 23 in the balloon catheter 60 according to this embodiment of the invention is that it is not essential for the linear distance measuring element 23 to be moved in one direction only, namely, in the distal direction in the direction of the arrow A from the reset position adjacent the proximal end of the catheter to the datum location 8 as in the case of the balloon catheter 1 described with reference to FIGS. 1 to 6. Since the signals received from the rotary encoder 65 are indicative of the direction of movement of the linear distance measuring element 23 along the catheter 3, once the linear distance measuring element 23 has been initially moved from the reset position adjacent the proximal end 4 of the catheter 3 of the balloon catheter 60, the position of the linear distance measuring element 23 on the catheter 3 of the balloon catheter 60 is always known to the signal processor 20, since the count of the secondary optically detectable bands 30 are summed while the linear distance measuring element 23 is being urged distally in the direction of the arrow A along the catheter 3 and are deducted while the linear distance measuring element 23 is being urged proximally in the direction of the arrow B along the catheter 3.

Otherwise, the balloon catheter 60 is similar to the balloon catheter 1 and its use in conjunction with the system 2 is likewise similar.

Referring now to FIG. 9, there is illustrated a balloon catheter according to another embodiment of the invention, indicated generally by the reference numeral 70. The balloon catheter 70 is substantially similar to the balloon catheter 1 described with reference to FIGS. 1 to 6 and to the balloon catheter 60 described with reference to FIGS. 7 and 8, and similar components are identified by the same reference numerals. The balloon catheter 70 is also suitable for use with the system 2. The main difference between the balloon catheter 70 and the balloon catheter 60 is that in the linear distance measuring housing 23 the optical encoder 32 has been omitted, and the primary and secondary optically detectable bands 28 and 30 have also been omitted from the catheter 3. However, a primary element comprising an abutment element 71 is located at the proximal end 4 of the catheter 3 of the balloon catheter 70, which indicates the reset position for the linear distance measuring element 23. In other words, when the linear distance measuring element 23 is abutting the abutment element 71, the linear distance measuring element 23 is in the reset position. The abutment element 71 is located at the proximal end 4 of the catheter 3 at a predefined distance from the proximal end 29 of the balloon 7 of the balloon catheter 70.

Additionally, in this embodiment of the invention the rotary encoder 65 is configured to produce signals which as well as being indicative of the direction of relative linear movement between the linear distance measuring element 23 and the catheter 3 of the balloon catheter 70 also produces signals indicative of the distance of relative movement between the linear distance measuring element 23 and the catheter 3. The signal processor 20 is configured to determine the linear distance along the catheter 3 of the linear distance measuring element 23 from the proximal end 29 of the balloon 7 of the balloon catheter 70 from the signals received from the rotary encoder 65 of the linear distance measuring element 23.

Otherwise, the balloon catheter 70 and its use in the system 2 is similar to that already described with reference to the balloon catheter 60 and the balloon catheter 1.

In the balloon catheters 1 and 60, the count of the secondary optically detectable bands 30 is zeroed in the signal processor 20 each time the linear distance measuring element 23 is urged into the reset position. In the case of the balloon catheter 70, the position of the linear distance measuring element 23 is zeroed in the signal processor 20 each time the linear distance measuring element 23 is urged into the reset position. This resetting of the count from or the position of the linear distance measuring element 23 typically would be done manually by providing a reset switch on the signal processor or on the linear distance measuring element 25, or in the case of the balloon catheter 70, a reset switch, such as a magnetic reed switch or other suitable switch means, could be provided in the linear distance measuring element 23 which would automatically send a reset signal to the signal processor 20 in response to the linear distance measuring element 23 abutting the abutment element 71 in the reset position.

Referring now to FIGS. 10 and 11, there is illustrated a portion of a balloon catheter according to another embodiment of the invention, indicated generally by the reference numeral 80, also for determining linear distance on the catheter 3 from the balloon 7 of the balloon catheter 80. The balloon catheter 80 is suitable for use with the system 2 described with reference to FIGS. 1 to 6. The balloon catheter 80 is substantially similar to the balloon catheter 1, and similar components are identified by the same reference numerals. The only difference between the balloon catheter 80 and the balloon catheter 1 is that a bypass means is provided for bypassing the balloon 7 in order to allow fluid to flow through a lumen, vessel, valve or sphincter or the like past the balloon 7 when the balloon 7 is inflated and blocking the lumen, vessel, valve or sphincter. In this embodiment of the invention the bypass means comprises a bypass lumen 81 which extends through the catheter 3 between a pair of ports 82 and 83, which communicate the bypass lumen 81 externally of the catheter 2. The ports 82 and 83 are located in the catheter 3 adjacent the proximal end 29 and the distal end 48, respectively of the balloon 7 externally of the balloon 7 for accommodating fluid past the balloon 7 through a lumen, vessel, valve or sphincter in which the balloon 7 when inflated is blocking.

Otherwise, the balloon catheter 80 and its use in the system 2 of FIGS. 1 to 5 is similar to that of the balloon catheter 1.

Needless to say, it will be appreciated that any other suitable bypass means for bypassing the balloon 7 may be provided, for example, a bypass lumen could be provided extending through the balloon 7 from the proximal end to the distal end thereof, or alternatively, the balloon 7 may be shaped so that when inflated an inwardly extending recess or groove would be formed which would extend longitudinally along the outer circumferential surface of the balloon 7 from the proximal end to the distal end thereof, and into the balloon 7 for accommodating the fluid to bypass the balloon. It is also envisaged that a bypass tube may be provided extending through the balloon from the proximal end to the distal end, or a bypass tube may be located on an outer surface of the balloon, and would extend from the proximal end to the distal end of the balloon.

Referring now to FIG. 12, there is illustrated a balloon catheter according to another embodiment of the invention, indicated generally by the reference numeral 90, for use with the system 2 of FIG. 1 for determining the distance of a location in the human or animal body, for example, a valve orifice of the aortic valve in the heart of a human subject as illustrated in FIGS. 5 and 6, from a datum location, for example, an entry point of the balloon catheter 90 into the human body, such as the datum location 8 in the leg of the human subject as illustrated in FIG. 5. The balloon catheter 90 is substantially similar to the balloon catheter 1 described with reference to FIGS. 1 to 6, and similar components are identified by the same reference numerals. The main difference between the balloon catheter 90 and the balloon catheter 1 is that firstly, the secondary optically detectable bands 30 are provided on the catheter 3 over substantially the entire length of the catheter 3 of the balloon catheter 90, and secondly, the primary optically detectable band 28, instead of being located adjacent the proximal end of the catheter 3, is located towards the proximal end 29 of the balloon 7, but spaced apart from the proximal end 29 of the balloon 7. The space between the primary optically detectable band 28 and the proximal end 29 of the balloon 7 defines the reset position for the linear distance measuring element 23. Accordingly, in this embodiment of the invention the optical encoder 32 in the linear distance measuring element 23 counts the secondary optically detectable bands 30 from the primary optically detectable band 28 as the linear distance measuring element 23 is urged in a proximal direction, namely, in the direction of the arrow B from the reset position, or as the catheter 3 is being urged through the linear distance measuring element 23 from the reset position, and the count of the number of secondary optically detectable bands 30 from the primary optically detectable band 28 is indicative of the distance of the linear distance measuring element 23 from the proximal end 29 of the balloon 7.

In use, initially only the balloon 7 of the balloon catheter 90 is urged into the arterial or other system of the human or animal body through the point of entry into the human or animal body, namely, the datum location. Once the balloon 7 has been entered into the human or animal body, with the portion of the catheter 3 between the proximal end 29 of the balloon 7 and the primary optically detectable band 28 extending from the human or animal body, the linear distance measuring element 23 is urged along the catheter 3 and is located on the catheter 3 between the distal end 29 of the balloon 7 and the primary optically detectable band 28, in other words, the linear distance measuring element 23 is located in the reset position on the catheter 3 at the datum location. The count of the secondary optically detectable bands 30 from the linear distance measuring element 23 is zeroed in the signal processor 20. The balloon catheter 90 is then urged through the linear distance measuring element 23 at the datum location and through the arterial or other system into the human or animal body, and as the catheter 3 is being urged through the bore 27 in the linear distance measuring element 23, the optical encoder 32 counts the secondary optically detectable bands 30 on the catheter 3 from the primary optically detectable band 28. The signal processor 20 reads signals from the optical encoder 32 which are indicative of the count of the secondary optically detectable bands 30 from the primary optically detectable band 28, which in turn is directly indicative of the distance of the linear distance measuring element 23 from the proximal end 29 of the balloon 7. Relevant procedures at the aortic valve, or other location in the human or animal subject at which the balloon is located are also carried out by the balloon catheter 90 as already described with reference to the balloon catheter 1 of FIGS. 1 to 6.

Otherwise, the balloon catheter 90 and its use are similar to the balloon catheter 1 described with reference to FIGS. 1 to 6.

While the linear distance measuring element of the balloon catheters 1 and 60 has been described as comprising an optical encoder, it will be appreciated that any other suitable encoder may be used, for example, a magnetic encoder, a capacitive encoder, and in which case, the primary and secondary detectable elements on the catheter would be provided by suitable elements. For example, in the case of a magnetic encoder the primary and secondary detectable elements could be provided by a magnetic element, and in the case of a capacitive encoder, the primary and secondary detectable elements could be provided by electrically conductive elements. Such primary and secondary detectable elements could be printed onto the catheter by suitable magnetic and/or electrically conductive inks, as appropriate. Needless to say, any other suitable detecting means for detecting the primary and secondary detectable elements may be used, and it will of course be appreciated that other suitable measuring means for measuring linear distance along the catheter may be provided.

It is also envisaged that instead of primary and secondary detectable elements, one or more barcodes may be provided on the catheter which would be read by a suitable reading device located in the linear distance measuring element, and it is envisaged that the barcode or barcodes would indicate the precise distance of that particular barcode or barcodes from the balloon, for example, from the proximal or distal end of the balloon or other reference point on the balloon. Further, it is envisaged that the primary and secondary detectable elements accompanied by numbers or encoded numbers indicative of the distance of each detectable element from the balloon, for example, from the proximal or distal end thereof, would be provided on the catheter, and a reading means in the linear distance measuring element would be adapted to read the distances directly from the catheter, and produce a signal to the signal processor indicative of the distance of the linear distance measuring element from the balloon.

It will be appreciated that while the balloon catheters described with reference to FIGS. 1 to 10 have been described with the linear distance measuring element being urged from the proximal-most detectable element in a generally distal direction towards the datum location in order to determine the distance of the datum location from the remote site in the human or animal body, in certain cases, it is envisaged that the linear distance measuring element would be located adjacent the datum location, and as the balloon catheter is being urged into the iliofemoral artery through the datum location, the optical encoder or the rotary encoder in the linear distance measuring element would count the number of secondary detectable elements from the distal-most detectable element as the catheter is being urged through the datum location. In which case, it is envisaged that the primary detectable element would be located distally on the catheter 3 between the proximal end of the balloon and the proximal end of the catheter with the secondary detectable elements being located proximally of the primary detectable element, in other words, between the primary detectable element and the proximal end of the catheter, as in the case of the balloon catheter 90 described with reference to FIG. 12.

It is also envisaged that the measuring element of the balloon catheters 1 and 60 could be provided with a reset switch for resetting the count of the secondary detectable elements in the optical encoder to zero when the linear distance measuring element is located in the reset position proximally of the primary detectable element, or in cases where the primary detectable element is located distally of the secondary detectable elements, when the linear distance measuring element is located in a reset position distally of the primary detectable element. It is also envisaged that in the case of the balloon catheter 70, the linear distance measuring element may be provided with a reset switch for producing a signal to the signal processor to reset the distance of the linear distance measuring element from the proximal end 29 of the balloon 7 in the signal processor 20 to zero when the linear distance measuring element is in the reset position abutting the abutment element 71. Needless to say, the reset switch could be provided on the linear distance measuring elements of the balloon catheters 1 and 60 for producing a signal to the signal processor 20 for zeroing the count of the secondary detectable elements in the signal processor 20 when the linear distance measuring element is in the reset position proximally of the primary detectable element, or in cases where the primary detectable element is located distally of the secondary detectable elements, as in the case of the balloon catheter 90 of FIG. 12, when the linear distance measuring element is located in a reset position distally of the primary detectable element.

While the balloon catheters and the system have been described for determining the transverse cross-sectional area and the diameter of the valve orifice of an aortic valve, and the distensibility index value thereof whereby the balloon catheter is entered into the subject through the iliofemoral artery in the leg of the subject, it is envisaged in certain cases that the balloon catheter may be entered transapically to the aortic valve, whereby the balloon catheter would be entered through the chest of the subject and through the wall of the heart to the aortic valve.

While the signals from the signal processor 20 have been described as being outputted to a laptop computer for displaying an image of the graphical representation of the longitudinal profile of the inflated balloon, and other data on a visual display screen of the laptop computer, it will be appreciated that signals from the signal processor may be applied to any suitable computer or computer system for displaying the image of the graphical representation of the longitudinal profile of the balloon and the data on any visual display screen controlled by that computer or computer system. Indeed, it is envisaged that in certain cases specific hardware and/or software may be provided in place of the signal processor and the laptop computer, which would include a visual display screen on which the image of the graphical representation of the longitudinal profile of the inflated balloon would be displayed. Further, it is envisaged that in certain cases the signal processor may be replaced by suitable software which would be loaded onto a computer, a laptop computer or any other computer system or hardware for producing the images of the graphical representation of the longitudinal profile of the inflated balloon and the other data on a suitable visual display screen.

It will also be appreciated that while the balloon catheters according to the invention have been described for determining the distance of an aortic valve from a datum location, namely, an entry site into the human body, the balloon catheters according to the invention may be provided for determining the linear distance of any remote site in a human or animal body from a datum location.

While the distensibility index values of the valve orifice of the aortic valve, and of the narrowed regions in the respective vessels have been described as being computed by dividing the respective computed transverse cross-sectional areas of the valve orifice and the narrowed regions of the vessels by the corresponding pressures of the inflating medium within the balloon, it is envisaged that the distensibility index values may be computed by dividing the diameters of the valve orifice and the narrowed regions by the corresponding pressures of the inflating medium within the balloon. The diameters of the valve orifice and the narrowed regions of the vessels would be derived from the corresponding computed transverse cross-sectional areas of the valve orifice and the narrowed regions, assuming the valve orifice and the narrowed regions to be of circular transverse cross-section.

It will also be appreciated that other methods for determining the distensibility index values of the valve orifice of an aortic valve or a narrow region in a lumen, vessel or artery or the distensibility index value of any other vessel, lumen or valve may be determined by the system according to the invention using other formulae for determining the distensibility index value.

1.-74. (canceled)

75. A balloon catheter comprising an elongated catheter, an inflatable balloon located on the catheter, and a linear distance measuring means located adjacent the catheter configured to measure linear distance on the catheter from the balloon.

76. A balloon catheter as claimed in claim 75 in which the linear distance measuring means is located on the catheter, and preferably, the linear distance measuring means is configured to accommodate relative linear movement between the catheter and the linear distance measuring means, and advantageously, the linear distance measuring means is configured to accommodate linear movement of the catheter through the linear distance measuring means, and preferably, the linear distance measuring means is slideably mounted on the catheter, and advantageously, the linear distance measuring means is responsive to relative linear movement between the catheter and the linear distance measuring means for determining the linear distance on the catheter from the balloon.

77. A balloon catheter as claimed in claim 75 in which a primary element is located on the catheter at a predefined distance from the balloon, the primary element defining a reset position of the linear distance measuring means, and preferably, the primary element is located at a predefined distance from one of the proximal end of the balloon and the distal end of the balloon, and advantageously, the primary element is located adjacent the proximal end of the catheter, and preferably, the primary element is located distally along the catheter between the proximal end of the catheter and the proximal end of the balloon, and advantageously, the primary element is located adjacent the proximal end of the balloon.

78. A balloon catheter as claimed in claim 75 in which a detecting means is provided for detecting relative linear movement between the catheter and the linear distance measuring means, and for producing a signal indicative of the distance of the linear distance measuring means from the balloon, and preferably, the detecting means is located on the linear distance measuring means.

79. A balloon catheter as claimed in claim 78 in which a plurality of longitudinally spaced apart secondary detectable elements are provided at predefined distances from the primary element and spaced apart therefrom, the secondary detectable elements being detectable by the detecting means for determining the distance of the linear distance measuring means from the balloon.

80. A balloon catheter as claimed in claim 79 in which the secondary detectable elements are equi-spaced apart longitudinally on the catheter, and preferably, the longitudinal spacing between the primary element and the adjacent one of the secondary detectable elements is similar to the spacing between the secondary detectable elements, and advantageously, the primary element comprises a detectable element detectable by the detecting means, and preferably, the detecting means is configured to count the ones of the secondary detectable elements detected by the detecting means as the one of the linear distance measuring means and the catheter is moved longitudinally relative to the other one of the linear distance measuring means and the catheter for producing a signal indicative of the distance of the linear distance measuring means along the catheter from the balloon, and preferably, the detecting means comprises an encoder, and advantageously, the encoder is configured to count the detected ones of the secondary detectable elements as the one of the linear distance measuring means and the catheter is moved longitudinally relative to the other one of the linear distance measuring means and the catheter, and preferably, the secondary detectable elements comprise respective ones of optically detectable elements, magnetically detectable elements, and capacitively detectable elements, and the detecting means comprises one of an optical encoder, a magnetic encoder and a capacitive encoder, and advantageously, the primary element comprises one of an optically detectable element, a magnetically detectable element and a capacitively detectable element.

81. A balloon catheter as claimed in claim 79 in which at least each secondary detectable element comprises an encoded element with the distance of the secondary detectable element from the balloon being encoded therein.

82. A balloon catheter as claimed in claim 81 in which each secondary detectable element comprises a barcode.

83. A balloon catheter as claimed in claim 77 in which the primary element comprises an encoded element with the distance of the primary element from the balloon being encoded therein, the encoded element being readable by the detecting means, and preferably, the encoded element of the primary element comprises a barcode.

84. A balloon catheter as claimed in claim 77 in which each one of the primary and secondary detectable elements extends around the catheter in a band-like configuration, and preferably, each one of the primary and secondary detectable elements extends completely around the catheter, and advantageously, at least the secondary detectable elements are printed onto the catheter by one of an optically detectable ink, a magnetic ink and an electrically conductive ink, and preferably, the primary element is printed onto the catheter by one of an optically detectable ink, a magnetic ink and an electrically conductive ink, and advantageously, a movement sensing means is located in the linear distance measuring means for detecting relative longitudinal movement between the linear distance measuring means and the catheter, and preferably, the movement sensing means is configured to detect the direction of relative movement between the linear distance measuring means and the catheter, and to produce a signal indicative of the direction of the relative movement, and preferably, the movement sensing means is configured to detect the distance moved by one of the linear distance measuring means and the catheter relative to the other one of the linear distance measuring means and the catheter, and advantageously, the movement sensing means comprises a rotatable element rotatably mounted in the linear distance measuring means and configured to be in rotatable engagement with the catheter, and a rotary encoder co-operable with the rotatable element for monitoring the direction of rotation of the rotatable element and for producing a signal indicative of the direction of relative movement between the linear distance measuring means and the catheter, and preferably, the rotary encoder of the movement sensing means is configured to determine distance of the linear distance measuring means from the balloon, and to produce a signal indicative of the linear distance along the catheter of the linear distance measuring means from the balloon, and advantageously, the primary element comprises an abutment element engageable with the linear distance measuring means for defining the reset position of the linear distance measuring means.

85. A balloon catheter as claimed in claim 75 in which a catheter accommodating bore extends through the linear distance measuring means, and the catheter is longitudinally slideable in the catheter accommodating bore, and preferably, the linear distance measuring means comprises a linear distance measuring element, and advantageously, the linear distance measuring element comprises a housing defining the catheter accommodating bore extending therethrough.

86. A balloon catheter as claimed in claim 75 in which a balloon measuring means is provided for producing signals indicative of one of the diameter and the transverse cross-sectional area of the balloon adjacent a plurality of longitudinally spaced apart locations intermediate the respective opposite proximal and distal ends of the balloon, and preferably, the balloon measuring means comprises an impedance planimetry measuring means, and advantageously, a pressure sensing means is provided for monitoring pressure within the balloon, and preferably, the catheter extends from the proximal end to a distal end, and the balloon is located adjacent the distal end of the catheter.

87. A balloon catheter as claimed in claim 75 in which a bypass means is provided in the balloon catheter adjacent the balloon for accommodating a fluid in a lumen, a vessel or a valve in which the balloon is located from one of the proximal and distal ends of the balloon to the other one of the proximal and distal ends thereof, and preferably, the bypass means comprises a bypass conduit extending between and communicating a pair of ports, the ports being located with the balloon located therebetween.

88. A balloon catheter as claimed in claim 87 in which the bypass conduit extends through the catheter, and the respective ports are located on the catheter, or alternatively, the bypass conduit extends through the balloon between a proximal end and a distal end thereof, the ports being located on the balloon adjacent the proximal end and the distal end thereof, or alternatively, the bypass means comprises an elongated groove defined by the balloon extending from the proximal end to the distal end thereof.

89. A system comprising the balloon catheter as claimed in claim 75, and a signal processor configured to read signals from the linear distance measuring means of the balloon catheter and for determining the distance along the catheter of the linear distance measuring means from the balloon.

90. A system as claimed in claim 89 in which the signal processor is configured to read signals from the balloon measuring means and for determining a location of the balloon at which one of the diameter and the cross-sectional area of the balloon is less than the corresponding ones of the diameters and the cross-sectional areas of the balloon adjacent the said location on respective opposite sides thereof with the balloon defining a waisted portion adjacent the said location, and preferably, the signal processor is configured to determine the distance of the linear distance measuring means from the location of the balloon at which the balloon defines the waisted portion, and advantageously, the signal processor is configured to produce a signal indicative of one or more of the distances along the catheter of the linear distance measuring means from the balloon, and the distance along the catheter of the linear distance measuring means from the location of the balloon at which the balloon defines the waisted portion, and preferably, the signal processor is configured to produce a signal indicative of a representation of the longitudinal profile of the balloon, and advantageously, the signal processor is configured to read signals from the pressure sensing means, and to determine the pressure within the balloon from the signals read from the pressure sensing means, and preferably, the signal processor is configured to produce a signal indicative of the value of the pressure in the balloon.

91. A system as claimed in claim 89 in which the signal processor is configured to compute a value of a distensibility index of a lumen, vessel or a valve orifice within which the balloon is located, and preferably, the signal processor is configured to compute the value of the distensibility index as a function of the transverse cross-sectional area of the balloon adjacent a location of the balloon adjacent which the value of the distensibility index of the lumen vessel or valve orifice is to be determined and the pressure within the balloon, and preferably, the signal processor is configured to produce a signal indicative of the value of the distensibility index.

92. A system as claimed in claim 89 in which the signals produced by the signal processor are adapted for applying to a visual display means for displaying data of which the signals are representative, and preferably, a visual display means is provided for displaying the data, and advantageously, the visual display means is configured to display a representation of a longitudinal profile of the balloon, and preferably, the signal processor is configured to produce the signals adapted for applying to a laptop computer, and advantageously, the signal processor comprises a microprocessor, and preferably, the signal processor comprises a computer.

93. A method for determining the linear distance of a remote site in a vessel, lumen, valve or sphincter within a human or animal body from a datum location, the method comprising:
inserting the balloon catheter as claimed in claim 75 into the human or animal body adjacent the datum location,
urging the balloon catheter through the human or animal body with the distal end thereof being the leading end until the balloon is located at the remote site,
inflating the balloon for retaining the balloon at the remote site,
with the linear distance measuring means adjacent the datum location operating a signal processor to read signals from the linear distance measuring means and to determine the linear distance along the catheter of the linear distance measuring means from the balloon in order to determine the distance of the remote site from the datum location.

94. A method as claimed in claim 93 in which the balloon is inflated, and the signal processor is operated to read signals from the balloon measuring means and to determine a location of the balloon at which one of the diameter and the transverse cross-sectional area of the balloon is less than the corresponding ones of the diameters and the transverse cross-sectional areas of the balloon adjacent the said location on opposite sides thereof with the balloon defining a waisted portion adjacent the said location, and preferably, the signal processor is operated to determine the linear distance along the catheter from the datum location to the location at which the balloon defines the waisted portion, and advantageously, the balloon is inflated at the remote site until the balloon defines the waisted portion, and preferably, the signal processor is operated to determine one of the diameter and the transverse cross-sectional area of the balloon adjacent the waisted portion defined by the balloon for determining one of the diameter and the cross-sectional area of the vessel, lumen, valve or sphincter adjacent the waisted portion of the balloon, and advantageously, the signal processor is operated to determine the pressure of inflating medium in the balloon, and preferably, the signal processor is operated to compute a value of the distensibility index of the vessel, lumen, valve or sphincter adjacent the waisted portion of the balloon, and advantageously, the remote site is a valve orifice in the human or animal body, and the balloon catheter is urged through the human or animal body until the balloon is located in the valve orifice and the balloon is inflated to define the valve orifice adjacent the waisted portion, and preferably, the valve orifice is a valve orifice of an aortic valve, and advantageously,
the remote site is a narrow region in a vessel or lumen, and the balloon catheter is urged through the human or animal body until the balloon is located in a narrow region of a vessel or lumen, and the balloon is inflated to define the narrow region adjacent the waisted portion.
